(19) United States
(12) Patent Application Publication
(10) Pub. No.: US 2011/0112050 A1
(43) Pub. Date: May 12, 2011

(54) SEPARATION OF POLYSACCHARIDES BY CHARGE DENSITY GRADIENT

(75) Inventors: Shmuel Bukshpan, Ramat Hasharon (IL); Gleb Zilberstein, Rehovot (IL)

(73) Assignee: CRYSTAL CLEAR PARTNERSHIP, Rehovot (IL)

(21) Appl. No.: 12/993,236

(22) PCT Filed: May 20, 2009
(86) PCT No.: PCT/IL09/00502

§ 371 Date: Jan. 18, 2011

Related U.S. Application Data
(60) Provisional application No. 61/054,495, filed on May 20, 2008.

(51) Int. Cl. A61K 31/727; C08B 37/10; A61P 1/00; A61P 25/28; A61P 7/00; A61P 3/00; B01D 57/02; B01D 61/42

(52) U.S. Cl. 514/56; 536/21; 204/550; 204/456; 204/543

(57) ABSTRACT

Methods and apparatus for the separation of polysaccharides, particular heparin products, and glycosylated molecules are provided. The separation is based on the molecular weight and charge, by application of an electric field across a low-friction matrix, modified with a charged separation agent comprising charged regions ordered in a monotonous sequence distributed throughout the matrix, to generate a charge density gradient formed when an external electric field is applied. Saccharides of different charges migrate differently across the porous matrix and immobilized by charge neutralization in different charge regions of the matrix.

### FIELD OF THE INVENTION

The present invention generally relates to methods and products associated with separating and analyzing heterogeneous populations of polysaccharides, particularly sulfated polysaccharides and low molecular weight heparin, by application of an electric field through a charge density gradient. The invention is further directed to polysaccharides and low molecular weight heparin preparations and pharmaceutical compositions comprising them for therapeutic uses.

### BACKGROUND OF THE INVENTION

Polysaccharides are polymeric carbohydrate structures, formed of repeating units (chains of monosaccharides) that are joined together by glycosidic bonds. The polysaccharide structures may be linear and/or branched. The linkage of the monosaccharides into chains may create chains of varying length, ranging from chains of two monosaccharides (disaccharides), to thousands of the monosaccharides. The polysaccharides have diverse roles within the biological processes. In general, they may be divided into several functional groups, such as: structural polysaccharides, storage polysaccharides, and the like. In addition, the polysaccharides may be combined with other molecules, such as, proteins or lipids to form other biological molecules. For example, peptidoglycans, which are a combination of protein and polysaccharide, can be found in the cell wall of certain bacteria. Glycolipids, which are a combination of polysaccharides and lipids, can be found in the cell membrane.

Heparin, which is a highly sulphated glycosaminoglycan (a long unbranched polysaccharide consisting of a repeating disaccharide unit), is produced by mast cells, and is a widely used clinical anticoagulant. Heparin is one of the first biopolymeric drugs and one of the few carbohydrate drugs. Heparin primarily elicits its anticoagulant effect through two mechanisms, both of which involve binding of antithrombin III (AT-III) to a specific pentasaccharide sequence contained within the polymer. In addition to its anticoagulant properties, its complexity and wide distribution in mammals have lead to the suggestion that heparin may also be involved in a wide range of additional biological activities (such as. interaction with growth factors, regulation of cell proliferation and angiogenesis, modulation of proteases and antiproteases, and the like).

Heparan-Sulfate (HS) is highly sulfated linear polysaccharide characterized by repeating units of disaccharides containing a uronic acid (glucuronic or iduronic) and glucoseamine, which is either N-sulfated or N-acetylated. Heparin is a specialized form of HS and differs from HS in the degree of modification of the sugar residues.

Although heparin is highly efficacious in a variety of clinical situations and has the potential to be used in many others, the side effects associated with heparin therapy are many and varied. For example, Un-fractionated Heparin (UFH) is produced by autodigestion of porcine mucosa rich in glycosaminoglycans and by mast cells. The molecular weight of UFH is between 2750 Da and 30000 Da. Due to its erratic pharmacokinetics following s.c. administration, UFH has been administered by intravenous injection instead. Additionally, the application of UFH as an anticoagulant has been hampered by the many side effects associated with non-specific plasma protein binding with UFH. Side effects such as heparin-induced thrombocytopenia (HIT) are primarily associated with the long chain of UFH, which provides binding domains for various proteins. Other side effects include intracranial hemorrhage, bleeding, internal/external hemorrhage, hepatic enzyme (AST and ALT) level elevation, and dermal lesion at the site of injection. This has led to generation and utilization of low molecular weight heparin (LMWH) as an efficacious alternative to UFH.

LMWH are produced from UFH by controlled chemical (nitrous acid or alkaline hydrolysis) or enzymatic (Heparinase) depolymerization and has a mean molecular weight of 4000-6500 Da and a chain length of 13-22 sugars. Compared to UFH, the LMWH are characterized by a longer plasma half-life time, a lower effect on platelets and endothelium, a higher bioavailability even at lower doses, and a lower rate of haemorrhagic diathesis at a similar anticoagulative effect. In addition to anticoagulant activity, LMWH was also suggested as inhibitor of Tumor necrosis factor alpha (TNFα) activity.

Although attention has been focused on LMWH as heparin substitutes due to their more predictable pharmacological action, reduced side effects, sustained antithrombotic activity, and better bioavailability, there is at present no means of correlating their activity with a particular structure or structural motif due to the structural heterogeneity of heparin and LMWH, as it has been technically unfeasible to determine their structures, and there has been no reliable and readily available means for providing consistent LMWH preparations or for monitoring LMWH levels in a subject.

Pharmaceutical preparations of these polysaccharides are heterogeneous in length and composition. As such, only a portion of a typical preparation possesses anticoagulant activity. At best, the majority of the polysaccharide chains in a pharmaceutical preparation of heparin or LMWH are inactive, at worst, these chains interact nonspecifically with plasma proteins to elicit the side effects associated with heparin therapy. Therefore, it is important to develop LMWH preparations having defined composition that retain the anticoagulant activity and other desired activities of UFH but have reduced side effects. LMWHs, essentially due to their reduced chains sizes and dispersity, display markedly less non-specific plasma protein binding. However, all LMWHs that are currently clinically available also possess reduced anti-IIa activity as compared to UFH. Because of this decreased activity, a larger dose of LMWH is required (compared to UFH) in order to achieve a similar anti-coagulant activity.

Moreover, the heterogeneity of heparin products is not only a difference between different Heparin products but also of different batches of the same product. For example, studies have shown that there is substantial variation between batches of commercially available LMWH (Lovenox™, Aventis).

The most widely used techniques for the separation and identification of biomolecules, biochemicals and other analytes involve gel electrophoresis. Currently used matrices for gel electrophoresis include polyacrylamide, agarose, gelatin or other gels formed of cross linked polymers or long chain polymers. Biomolecules such as nucleic acids (DNA and RNA) and proteins exhibit a correlation between their mass and their charge. This allows the separation by size of such biomolecules across an electric field. In polyacrylamide gel electrophoresis (PAGE), charged proteins are separated in polyacrylamide gels based on their size (molecular mass) in native and denatured form. Various types of polyacrylamide gels exist, that vary in the degree of cross-linking and the nature of the denaturing surfactant included in the gel. The surfactant having the most widespread use is sodium dodecyl sulfate (SDS).

Another conventional electrophoretic separation method is isoelectric focusing (IEF), a special technique for separating amphoteric substances such as peptides and proteins in an electric field, across which there is both a voltage and a pH gradient, acidic in the region of the anode and alkaline near the cathode. Each substance in the mixture will migrate to a position in the separation column where the surrounding pH corresponds to its isoelectric point. There, in zwitterion form with no net charge, molecules of that substance cease to move in the electric field. Different amphoteric substances are thereby focused into narrow stationary bands.

Another method commonly used for protein separation based on the charge of the protein is ion exchange chromatography (IEC). In IEC, charged substances are separated via column materials that carry an opposite charge. The ionic groups of exchanger columns are covalently bound to the gel matrix and are compensated by small concentrations of counter ions, which are present in the buffer. When a sample is added to the column, an exchange with the weakly bound counter ions takes place. The IEC principle includes two different approaches: anion exchange and cation exchange according to the charge of the ligands on the ion exchange resin.

An additional method for protein separation by the size of the protein is size exclusion chromatography. This method, also known as gel filtration (GPC) or molecular-sieve chromatography, is based on the different size and shape of proteins. Proteins of different sizes penetrate into the internal pores of the beads to different degrees. Small protein molecules are retarded by the column while large molecules pass through more rapidly.

Additional methods for protein separation may include Capillary Zone Electrophoresis and electrochromatography.

In contrast to biomolecules such as proteins and nucleic acids, which exhibit a correlation between their mass and their charge, polysaccharides and other glycosilated molecules, such as, for example, glycoproteins, do not exhibit such correlation.

Available methods for qualitative and quantitative analysis and separation of polysaccharide enable low/high resolution molecular weight analysis of the different Heparin fragments within the sample or low resolution preparative separation of the sample. Yet, these methods are not capable of high resolution preparative separation of the preparation. Current methods of LMWH preparation lack standardization and result in preparations that may vary substantially from batch to batch in composition and in efficacy. In an attempt to characterize the molecular, structural, and activity variations of heparin, several techniques have been investigated for the analysis of heparin preparations. Gradient polyacrylamide gel electrophoresis (PAGE) and strong ion exchange HPLC (SAX) have been used for the qualitative and quantitative analysis of heparin preparations. Although the gradient PAGE method can be useful in determining molecular weight, it suffers from a lack of resolution, particularly the lack of resolution of different oligosaccharides having identical size. SAX-HPLC, which relies on detection by ultraviolet absorbance, is often insufficiently sensitive for detecting small amounts of structurally important heparin-derived oligosaccharides. Other methods such as Matrix Assisted Laser Desorption Mass Spectrometry with Time of Flight Mass Spectrometry (MALDI-TOF-MS), have very high resolution, yet these methods are not preparative.

As current technologies for analyzing heparins and other glycosaminoglycans are insufficient, it has been heretofore impossible to create LMWH preparations with any degree of batch-batch consistency, or to predict the potency of a given batch. Moreover, there is no preparative method that will allow composing different and specific heparin mixtures that will retain the anticoagulant activity and other desired activities of heparin but will have reduced side effects.

There is thus an unmet need in the art for methods for the separation, utilization and characterization of polysaccharides and particularly of heparin fragments and LMWHs which are efficient, cost-effective and which can be utilized to polysaccharides of a wide molecular weight, size and length range, and which can be adapted to large scale (e.g., purification) and/or automated. There is also a widely recognized unmet need for providing purified and characterized low molecular weight heparins useful as anticoagulants lacking side effects, and for the inhibition and prevention of the proinflammatory cytokine cascade, induced by TNFα in autoimmune diseases, neurodegenerative disorders and inflammation mediated pathological conditions.

### SUMMARY OF THE INVENTION

It was now unexpectedly found that separation on a charge density gradient matrix is also suitable for separating polysaccharides which are separated based on their charge.

According to some embodiments, there is provided a high resolution separation and analysis method that enable analytical and preparative separation of polysaccharides, glycoproteins, recombinant proteins, and the like, or any combination thereof. In particular, there is provided a high resolution separation and analysis method that enable analytical and preparative separation of heparin fragments and low molecular weight heparin (LMWH). Such separation and analysis method, which is based on controlling the electrophoretic mobility of the analytes in a charge density gradient matrix, enable design of specific defined LMWH preparations. These preparations are designed to retain the anticoagulant activity, anti inflammatory activity and other desired activities of heparin but have reduced side effects. In addition, the separation and analysis method enable quality control of heparin preparation and reduce variations.

According to some embodiments, there are provided methods for analytical and preparative separation of polysaccharides, glycoproteins, recombinant proteins, and the like, or any combination thereof. In particular, there are provided methods for analytical and preparative separation of LMWH, purified according the method, and prophylactic and therapeutic uses of the purified LMWH.

According to one aspect, the present invention provides at least one polysaccharide separated on the basis of their charge, using a method comprising subjecting a charged polysaccharide to an electric field using a matrix (preferably a low friction matrix) comprising a charged separation agent.

According to a preferred embodiment, the polysaccharide is a LMWH.

According to one embodiment, the at least one polysaccharide is separated using a method comprising: a) providing a preparation comprising at least one charged polysaccharide; and b) subjecting the at least one charged polysaccharide to an electrical field using a matrix comprising a charged separation agent wherein the polysaccharides are separated according to their charge.

According to another embodiment, the at least one polysaccharide is separated using a method comprising: a) providing a preparation comprising at least one charged polysaccharide; b) contacting the preparation comprising at least one charged polysaccharide with a matrix (e.g., a low-friction gel) comprising a charged separation agent having an opposite charge to that of the polysaccharides; and c) applying an electric field across the matrix.

According to one embodiment, the matrix comprises stable, spatially distributed charged regions ordered in a monotonous order preserving sequence, preferably starting with low charge and low charge density regions and ending with high charge regions. The charge density range in the matrix overlaps with that of an oppositely charged polysaccharide. When an external electric field is applied to a sample of the charged polysaccharide deposited at the low charge end of the matrix, the polysaccharide move through the different charged regions and focusing (immobilization by charge neutralization) of different polysaccharides in different regions occur.

According to some embodiments, the matrix is selected from the group consisting of: a polymeric gel, porous glass or other porous media, polymeric beads immobilized in compartments by porous membranes, and high viscosity liquids immobilized in compartments by porous membranes.

According to another aspect, pharmaceutical compositions comprising polysaccharides separated according to the method of the present invention, further comprising a pharmaceutically acceptable diluent or carrier, are provided.

According to some embodiments, the pharmaceutical compositions comprise at least one LMWH preparation purified on the basis of its charge, using a method comprising subjecting a preparation comprising at least one charged polysaccharide to an electric field using a matrix (preferably a low friction matrix) comprising a charged separation agent.

The choice of the pharmaceutical additives, carriers, diluents, excipients and the like, will be determined in part by the particular active ingredient, as well as by the particular route of administration of the composition. The routes of administration include but are not limited to oral, aerosol, parenteral, topical, ocular, transdermal, subcutaneous, intravenous, intramuscular, intraperitoneal, intrathecal, rectal and vaginal systemic administration. In addition, the pharmaceutical compositions of the invention can be directly delivered into the central nervous system (CNS) by intracerebroventricular, intraparenchymal, intraspinal, intracisternal or intracranial administration.

The pharmaceutical compositions can be in a liquid, aerosol or solid dosage form, and can be formulated into any suitable formulation including, but not limited to, solutions, suspensions, micelles, emulsions, microemulsions, aerosols, powders, granules, sachets, soft gels, capsules, tablets, pills, caplets, suppositories, creams, gels, pastes, foams and the like, as will be required by the particular route of administration.

According to yet another aspect, prophylactic and therapeutic uses of polysaccharides, particularly LMWH, separated on the basis of their charge are provided. According to this aspect, methods of prevention and treatment pathological conditions are provided, comprising administering to a subject in need thereof a pharmaceutical composition comprising at least one polysaccharide separated or characterized by a method involving subjecting a preparation comprising at least one charged polysaccharide to an electric field using a matrix (preferably a low friction matrix) comprising a charged separation agent.

According to certain embodiments, the invention includes methods for treating or preventing a condition in a subject wherein the subject has or is at risk of a disorder selected from the group consisting of disease associated with coagulation, such as thrombosis, cardiovascular disease, vascular conditions or atrial fibrillation; migraine, atherosclerosis; an inflammatory disorder, such as autoimmune disease or atopic disorders; an allergy; a respiratory disorder, such as asthma, emphysema, adult respiratory distress syndrome (ARDS), cystic fibrosis, or lung reperfusion injury; a cancer or metastatic disorder; an angiogenic disorder, such as neovascular disorders of the eye, osteoporosis, psoriasis, and arthritis; Alzheimer's; bone fractures such as hip fractures; or is undergoing or having undergone surgical procedure, organ transplant, orthopedic surgery, hip replacement, knee replacement, percutaneous coronary intervention (PCI), stent placement, angioplasty, coronary artery bypass graft surgery (CABG). The compositions of the invention are administered to a subject having or at risk of developing one or more of the diseases in an effective amount for treating or preventing the disease.

According to additional embodiments, the invention provides purified and characterized LMWHs, as inhibitors of the TNFα proinflammatory cytokine cascade. According to some embodiments, there are provided therapeutic uses of LMWH produced or characterized according to the method of the present invention, as inhibitors of the proinflammatory cytokine cascade for inhibiting, preventing or ameliorating the development of conditions associated with inflammation or in response to viral or bacterial infections.

In some embodiments, the present invention provides a method for the inhibition of proinflammatory cytokine cascade, for treatment of cytokine mediated inflammatory conditions which arise in response to infection with a virus or a bacteria, and for prevention, amelioration or treatment of inflammation, fibrosis and vasculopathy caused by irradiation which comprises administering to a patient in need thereof a pharmaceutical composition comprising an effective amount of a defined preparation of LMWH. The method comprises treating the patient with a pharmaceutical composition comprising a preparation of LMWH, purified and/or characterized by the method of the present invention, in an amount sufficient to inhibit the inflammatory cytokine cascade, wherein the patient is suffering from, or at risk for, a condition mediated by the inflammatory cytokine cascade.

According to embodiments of the present invention, any condition, mediated by TNFα is potential for being treated with a pharmaceutical composition comprising a LMWH prepared or analyzed according to the method of the present invention. According to one embodiment, the condition mediated by the TNFα, which may be treated by a pharmaceutical composition comprising a LMWH prepared or analyzed according to the method of the present invention, is selected from the group consisting of: inflammatory bowel disease, ulcerative, acute or ischemic colitis, Crohn's disease and cachexia (wasting syndrome). According to another embodiment, the condition involves a bacterial infection. According to a specific embodiment the condition is septic shock (sepsis, endotoxic shock) or disseminated bacteremia. According to yet another embodiment, the condition is a neurodegenerative disorder. According to a specific embodiment the neurological disorder is selected from the group consisting of Alzheimer's disease (AD), neurological lesions associated with diabetic neuropathy, demyelinating disorders other than autoimmune demyelinating disorders, retinal degeneration, muscular and glaucoma. According to a specific embodiment the TNFα mediated condition to be treated according to the invention is glaucoma, in which the compounds administered inhibit the TNFα mediated neural injury.

In another embodiment, the pharmaceutical composition comprising a LMWH according to the invention is for prevention and treatment of local or generalized inflammation condition initiated by infection with viruses or bacteria.

According to a specific embodiment the viral infection is selected from the group consisting of: influenza, respiratory syncytial virus infection, herpes infection and varicella zoster (shingles). According to another specific embodiment the bacteria is Propionibacterium acnes and LMWH preparation is used for treatment of Acne or Rosacea.

According to another embodiment of the present invention, the medicament comprising a LMWH, is administered to the subject in need thereof following development of a fulminant infection with herpes virus or with the varicella zoster virus (which causes shingles) or with the chicken pox virus.

The invention further provides defined and consistent preparations of polysaccharides, particularly of LMWHs, that have enhanced properties as compared to the current generation of commercially available LMWHs, as well as methods for preparing and using such preparations.

In another aspect, the invention relates to selecting a safer, less variable LMWH to use for treating a patient, by determining and separating polysaccharides having desired activity, excluding other polysaccharides which are known to posses undesired activities.

According to some embodiments, the invention also relates to a method for broadening the therapeutic utility of heparins, LMWHs or synthetic heparins for use in areas other than as modulators of hemostasis, by understanding the mechanism of action of specific, individual components of specific heparins, LMWHs or synthetic heparins by separating and analyzing specific components and the effect those components can have in the treatment of a specific disease.

According to some embodiments, the invention also relates to broadening the therapeutic utility of heparins, LMWHs or synthetic heparins for treating clot bound thrombin by designing novel LMWHs of smaller sizes, and/or of increased anti-IIa activity that are active and can reach and treat the thrombus.

According to some embodiments, the invention also relates to a method for designing heparins, LMWHs or synthetic heparins with ideal product profiles including, but not limited to such features as high activity, having both anti-Xa and anti-IIa activity, having a desired ratio between the anti-Xa and anti-IIa activity, titratable, well characterized, neutralizable, lower side effects including reduced HIT, attractive pharmacokinetics, and/or reduced PF4 binding that allow for optional monitoring and can be practically manufactured by separating and analyzing the activity of specific components of a composition that includes a mixed population of polysaccharides, such as glycosaminoglycans (GAGs), HLGAGs, UFH, FH, LMWHs, or synthetic heparins including but not limited to enoxaparin (Lovenox™); dalteparin (Fragmin); certoparin (Sandobarin™); ardeparin (Normiflo); nadroparin (Fraxiparin™); parnaparin (Fluxum™); reviparin (Clivarirform); tinzaparin (Innohep™ or Logiparin), or Fondaparinux (Arixtra™) and enriching for components with desired activities and de-enriching for components with undesirable activities.

According to some embodiments, the invention also relates to novel heparins purified and/or characterized by the methods of the invention, such as, for example, novel heparins, LMWHs or synthetic heparins with desired product profiles, including, but not limited to such features as high activity, both anti-Xa and anti-IIa activity, having a desired ratio between the anti-Xa and anti-IIa activity, titratability, well characterized, neutralizable (e.g. by protamine), reduced side effects including reduced HIT, and/or attractive pharmacokinetics, that allow for optional monitoring, and novel heparins, LMWHs or synthetic heparins with different or enhanced anti-IIa activities. Thus in one aspect, the invention includes a LMWH preparation having an increased or decreased ratio of anti-IIa activity and anti-Xa activity, e.g., a LMWH preparation made by the methods described herein. In another aspect, the invention includes a panel of two or more LMWH preparations having different ratios of anti-IIa activity and anti-Xa activity, e.g., LMWH preparations made by the separation and analysis methods described herein.

In another aspect, the invention also includes a LMWH preparation prepared, purified or characterized by the methods described herein, e.g., a LMWH preparation comprising polysaccharides of specific size and charge.

The invention provides, in yet another aspect, use of at least one polysaccharide, prepared or characterized according to the method of the present invention, for preparation of a medicament for prevention or treatment of a disorder selected from the group consisting of disease associated with coagulation, such as thrombosis, cardiovascular disease, vascular conditions or atrial fibrillation; migraine, atherosclerosis; an inflammatory disorder, such as autoimmune disease or atopic disorders; an allergy; a respiratory disorder, such as asthma, emphysema, adult respiratory distress syndrome (ARDS), cystic fibrosis, or lung reperfusion injury; a cancer or metastatic disorder; an angiogenic disorder, such as neovascular disorders of the eye, osteoporosis, psoriasis, and arthritis; Alzheimer's; bone fractures such as hip fractures; or is undergoing or having undergone surgical procedure, organ transplant, orthopedic surgery, hip replacement, knee replacement, percutaneous coronary intervention (PCI), stent placement, angioplasty, coronary artery bypass graft surgery (CABG); or for prevention or treatment of a condition involved over expression of TNFα.

Embodiments of the present invention are based on a novel principle for separating polysaccharides by controlling the electrophoretic mobility of the analytes in a matrix (e.g., a polymeric gel, porous glass, other porous media, polymeric beads immobilized in compartments by porous membranes, and high viscosity liquids immobilized in compartments by porous membranes) modified with a charged separation agent. The matrix comprises stable, spatially distributed charged regions ordered in a monotonous order preserving sequence, preferably starting with low charge and low charge density regions and ending with high charge regions. The charge density range in the matrix overlaps with that of an oppositely charged polysaccharides. When an external electric field is applied to a sample of the charged polysaccharide deposited at the low charge end of the matrix, the molecules will move through the different charged regions and focusing (immobilization by charge neutralization) of different polysaccharides in different charge regions will occur.

As opposed to conventional separation techniques such as polymeric gels, in which separation is based on the dependence of the migration velocity (mobility) on size and friction, the separation principle of the present invention is based on the total charge of the polysaccharide.

This principle of charge neutralization for trapping specifically charged species is the principle of operation of ion exchange columns. The present invention is based on the surprising discovery that this concept can be applied to conventional separation systems such as gel electrophoretic systems to generate novel matrices for separating polysaccharides based on their total charge. Such separation systems have not previously been described. Thus, according to one aspect, the present invention provides a method for the separation of polysaccharides, by subjecting a preparation comprising at least one charged polysaccharide to an electric field using a matrix (preferably a low friction matrix) comprising a charged separation agent, wherein the polysaccharides are separated on the basis of their charge.

In one embodiment, the present invention provides a method for the separation of polysaccharides by a) providing a preparation comprising at least one charged polysaccharide; and b) subjecting the at least one charged polysaccharide to an electric field using a matrix comprising a charged separation agent, wherein the polysaccharides are separated according to their charge.

In another embodiment, the present invention provides a method for the separation of polysaccharides by a) providing a preparation comprising at least one charged polysaccharide; b) contacting the preparation comprising at least one charged polysaccharide with a matrix (e.g., a low-friction gel) comprising a charged separation agent having an opposite charge to that of the polysaccharides; and c) applying an electric field across the matrix.

In yet another embodiment, the present invention relates to a method for controlling the electrophoretic mobility of polysaccharides for improving the separation of the polysaccharides, by subjecting a preparation comprising at least one charged polysaccharide to an electric field using a matrix comprising a charged separation agent, wherein the polysaccharides are separated according to their charge.

In another embodiment, the present invention relates to a system for the separation of polysaccharides according to their charge, the system comprising a matrix modified with a charged separation agent.

In one embodiment, the charged separation agent has an opposite charge to that of the polysaccharides. In another embodiment, the matrix is a porous polymeric gel, for example a polyacrylamide gel. In yet another embodiment, the analytes are separated by electrophoresis.

In accordance with a preferred embodiment, the charged separation agent is distributed throughout the polymeric gel so as to create a charge density gradient. The gradient is created by distributing charged regions in a monotonous order preserving sequence, preferably starting with low charge and charge density regions and ending with high charge. Alternatively, the charged species is constantly (evenly) distributed throughout the matrix.

In another embodiment, the present invention provides a method for the separation of polysaccharides, comprising the step of subjecting a preparation comprising at least one charged polysaccharide to gel electrophoresis using a polymeric gel comprising a charged separation agent, wherein the analytes are separated on the basis of their charge.

In another embodiment, the present invention provides a method for the separation of polysaccharides by a) providing a preparation comprising at least one charged polysaccharide; and b) subjecting the charged polysaccharide to an electric field using a polymeric gel comprising a charged separation agent, wherein the analytes are separated according to their charge.

In yet another embodiment, the present invention provides a method for the separation of polysaccharides by a) providing a preparation comprising at least one charged polysaccharide; b) contacting the preparation with a polymeric gel comprising a charged separation agent having an opposite charge to that of the polysaccharide; and c) applying an electric field across the gel.

According to specific embodiments, the methods for separating polysaccharides comprise at least one an additional step of extracting the separated polysaccharides from the matrix. The extraction can be performed by any method known in the art, including but not limited to extraction by salt, degrading the matrix, and dissolving the matrix.

In yet another embodiment, the present invention relates to a method for controlling the electrophoretic mobility of polysaccharides for improving the separation of the polysaccharides, by subjecting a charged preparation comprising at least one polysaccharide to an electric field using a polymeric gel comprising a charged separation agent, wherein the polysaccharides are separated according to their charge.

In another embodiment, the present invention relates to a gel system for the separation of polysaccharides according to their charge, the gel system comprising a polymeric gel modified with a charged separation agent.

When the matrix is a polymeric gel, the methods of the present invention can use any type of gel known in the art. In accordance with a preferred embodiment, the polymeric gel is a polyacrylamide gel. However, other gels can also be used, for example agarose gels, composite polyacrylamide-agarose gels, gelatins and the like. One of the advantages of the present invention is that it favors the use of low density gels to minimize the friction and enable focusing of large polysaccharides in a relatively short time. Suitable gels for this type of separation include but are not limited to low percentage polyacrylamide (e.g., equal to or less than about 5%) or composite acrylamide agarose gels (e.g., about 2%-5% acrylamide and about 0.5%-1% agarose).

Another important property of the proposed separation method is the realization that the resolution of a separated band is independent of the dimension of the initial packet and depends only on the gradient of the charge distribution in the separation medium (gel). This property removes the requirement of adding a stacking gel for band compression as generally used in standard SDS-PAGE. Diffusion effects which strongly influence the final dimension of the separated bands in conventional SDS-PAGE, are absent in the new method due to the focusing process.

Generally, the charged separation agent (also referred to herein as charged separation media) is a material which is either positively charged (cationic) or negatively charged (anionic), and can typically be any material that is commonly used in ion exchange separation techniques (i.e., ion exchange resins). Alternatively, the separation agent can be acrylamido derivatives used for the preparation of isoelectric focusing strips (immobilines).

Suitable gels for use in the methods of the present invention include, but are not limited to, slab gels, planar gels, capillary gels, in-tube gels, gels in discrete channels (e.g., a gel channel in a solid matrix), separation columns or any other geometry which preserves the charge distribution so that a charge density gradient can be generated. This enables a design where the linear charge resolution can be optimized for different charge regions.

Other suitable media or substrates for use in the methods and systems of the present invention are porous media on which charged anionic or cationic species can be immobilized (porous glass etc.) or high viscosity liquids immobilized in compartments by porous membranes. Another suitable medium can comprise porous polymer beads incorporating the charged separation agent e.g., ion exchange beads) and placed in compartments separated by a porous membrane.

The novel methods according to embodiments of the present invention remove most of the limitations of the standard separation techniques, both by extending the charge range to the region of low and high analyte sizes and by improving the charge determination accuracy in the whole range. The advantages of the methods of the present invention over conventional separation systems include: 1) replacement of the logarithmic scale with a pre-designed charge scale (e.g., linear) for improved accuracy of charge determination; 2) extension of the charge range into low and high charge analytes; 3) no diffusion effects; 4) no dependence of separated band width on initial packet dimensions; 5) the gel density used in this application is preferably very low which facilitates the separation process (faster drift velocity); 6) no need for gradient gels; 7) no need for stacking gel; 8) cost-effectiveness; and 9) due to the large abundance of ion exchange resins and other charged separation media such as immobilines, the methods of the invention are easy to use, and can be utilized to separate a large variety of analytes of a wide range of mass, charge, size or length.

According to some embodiments, there are thus provided new and versatile method and matrices for the separation of polysaccharides using separation techniques such as electrophoresis. They are suitable for planar, capillary in-tube electrophoresis, as well as multi-channel arrays of capillaries filled with charge gradient gels, serial arrays of discrete compartments with charge density overlapping a narrow charge range, arrays in a chip format (which can be automated), pre-designed charge focusing arrays for diagnosis, multi compartment trapping devices for scale up (purification) and other separation systems using other low friction media, under widely different conditions.

The availability of many types of charged ion-exchange resins and other charged materials which can be incorporated as charged separation media into gels and other porous media, allows for the extensive use of systems of the invention for separating a large variety of polysaccharides. Most importantly, the ability to generate pre-designed separation gradients provides a tremendous advantage over currently practiced methods, and enables the efficient separation of polysaccharides at very high or very low molecular weights, size and length. In these ways and others, the systems of the present invention are superior to conventional separation systems currently in use.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present disclosure will be understood and appreciated more fully from the following detailed description taken in conjunction with the appended drawings:

FIG. 1: Separation pattern of LMWH (Enoxiparin and Tinzapin) on gradient charged electrophoresis resolving gel;

FIGS. 2a-b: Separation of LMWH fractions obtained from Size Exclusion Chromatography (SEC);

FIG. 3: Schematic drawing of a Multicompartment mass fractionation device, according to some embodiments;

FIGS. 4a-b: Schematic drawing of a multicompartment charge fractionation device based on charged liquid compartments, according to some embodiments;

FIG. 5: Schematic drawing of a multicompartment charge fractionation device based on selective charge trapping in PA immobiline beads according to some embodiments; and

FIG. 6: Schematic drawing of a chip form multicompartment charge fractionation device.

### DETAILED DESCRIPTION OF THE INVENTION

According to some embodiments, there is thus provided a method for separation of polysaccharides, such as, for example UFH or LMWH's, based on migration of the polyanionic molecules in a polycationic polyacrylamide gel, made by incorporating positively-charged monomers into the neutral polyacrylamide backbone. Separation is obtained due to differential charge modulation of the various LMWH fragments that causes differential migration of the polyanionic molecules in the charge density gradient matrix under electric field based on immobilization by charge neutralization. The method may further be used for the separation and analysis of other biomolecules, such as, for example, glycoproteins, recombinant proteins, and the like.

This methodology enables complete solution for heparin separation, analysis, preparation and quality control:
i. High (fine) resolution charge separation of heparin preparation—revealing the different fragments contained in the preparation using the gradient charged electrophoresis resolving gel.
ii. Preparative separation of each fragment in a compartment charged gradient capillary trapping devices:
   The preparative separation enable sufficient amount of heparin fragments for further analysis. The fragments are tested in-vitro and in-vivo for their specific biologically activity.
   Based on the activity of each fragment it is possible to design specific anticoagulants drugs with enhanced activity, bioavailability yet with reduces side-effects
iii. Large scale preparative separation of the chosen fragment for a designed drug using a compartment charged gradient capillary trapping devices.
iv. Quality control of final preparation using the high resolution gradient charged electrophoresis resolving gel.

Unlike other biomolecules, such as, for example, nucleic acids and proteins, which exhibit a correlation between their mass and their charge (hence, enabling their separating by size in an electric field), polysaccharides and glycoproteins do not exhibit such a correlation. Thus, the embodiments of the present disclosure represents a marked improvement over existing techniques and appears as a valuable technique for analytical and preparative separation of any polysaccharides, in particular heparin and LMWHs and additional biomolecules, such as, for example, glycoproteins and various recombinant proteins.

A “polysaccharide” as used herein is a polymer composed of monosaccharides linked to one another. In many polysaccharides, the basic building block of the polysaccharide is actually a disaccharide unit, which can be repeating or non-repeating. Thus, a unit when used with respect to a polysaccharide refers to a basic building block of a polysaccharide and can include a monomeric building block (monosaccharide) or a dimeric building block (disaccharide). Polysaccharides include but are not limited to heparin-like glycosaminoglycans, chondroitin sulfate, hyaluronic acid and derivatives or analogs thereof, chitin in derivatives and analogs thereof, e.g., 6-O-sulfated carboxymethyl chitin, immunogenic polysaccharides isolated from phellinus linteus, PI-88 (a mixture of highly sulfated oligosaccharide derived from the sulfation of phosphomannum which is purified from the high molecular weight core produced by fermentation of the yeast pichia holstii) and its derivatives and analogs, polysaccharide antigens for vaccines, and calcium spirulan (Ca-SP, isolated from blue-green algae, spirulina platensis) and derivatives and analogs thereof.

As used herein the term “heparin” refers to polysaccharides having heparin-like structural and functional properties. Heparin includes, but is not limited to, native heparin, low molecular weight heparin (LMWH), heparin, biotechnologically prepared heparin, chemically modified heparin, synthetic heparin, and heparan sulfate. The term “biotechnological heparin” or “biotechnologically prepared heparin” encompasses heparin that is prepared from natural sources of polysaccharides which have been chemically modified and is described in Razi et al., Bioche. J. 1995 Jul. 15; 309 (Pt 2): 465-72. Chemically modified heparin is described in Yates et al., Carbohydrate Res (1996) November 20; 294: 15-27, and is known to those of skill in the art. Synthetic heparin is well known to those of skill in the art and is described in Petitou, M. et al., Bioorg Med Chem Lett. (1999) April 19; 9 (8): 1161-6. Native heparin is heparin derived from a natural source (such as porcine intestinal mucosa).

A polysaccharide according to the invention can be a mixed population of polysaccharides, e.g., heparin, synthetic heparin, LMWH preparation, or any combination thereof.

In some embodiments, the polysaccharide preparation is derived from a human or veterinary subject, an experimental animal, a cell, or any commercially available preparation of polysaccharides, such as, UFH or LMWH, including but not limited to enoxaparin (Lovenox™); dalteparin (Fragmin™); certoparin (Sandobarin™); ardeparin (Normiflo™); nadroparin (Fraxiparin™); parnaparin (Fluxum™); reviparin (Clivarin™); tinzaparin (Innohep™ or Logiparin), or fondaparinux (Arixtra™).

In a preferred embodiment, the heparin composition is digested, for example, chemically and/or enzymatically, either completely or incompletely. The enzymatic digestion may be carried out with a heparin degrading enzyme, such as, for example, heparinase I, heparinase II, heparinase III, heparinase IV, heparanase or functionally active variants and fragments thereof. The chemical digestion may be carried out with a chemical agent, such as, for example, oxidative depolymerization, e.g., with H₂O₂ or Cu+ and H₂O₂, deaminative cleavage, e.g., with isoamyl nitrite or nitrous acid, eliminative cleavage, e.g., with benzyl ester, and/or by alkaline treatment.

The present disclosure is based on the discovery of a novel separation matrix for separating polysaccharides. The matrix (for example, a low-friction matrix) is composed of a medium, such as a polymeric gel or another suitable porous medium such as porous glass, porous polymer beads immobilized in compartments by porous membranes and/or a viscous liquid immobilized in a porous membrane compartment modified with a charged separation agent, which is distributed across the matrix in charged regions (which can be continuous or discrete) ordered in a monotonous order preserving sequence, preferably starting with low charge and charge density regions and ending with high charge. A preparation comprising at least one charged polysaccharide is loaded onto the matrix, preferably at its low charge end. When an external electric field is applied, the polysaccharide migrates through the different charged regions and focusing (immobilization by charge neutralization) of different analytes in different charge regions will occur. The separation principle of the present disclosure is based on the total charge of the polysaccharide. Since different polysaccharides possess different charges, they will migrate differently across the matrix, thereby achieving separation. This overcomes the lack of correlation between the mass and the charge of the polysaccharide.

In accordance with a preferred embodiment, the charged separation agent is distributed throughout the matrix so as to create a charge density gradient. Preferably, the gradient is created by distributing the charged regions in a monotonous (continuous or discrete) sequence across the gel. The term “monotonous” means ordered and gradual increase or decrease in the charge density gradient. The gradient preferably starts with low charge density regions and ends with high charge density regions.

An alternative embodiment of charge distribution in a matrix is represented by a constant distribution of the charged species through the matrix. When biomolecules are electrophoretically driven through such a charged matrix each polysaccharide will acquire an effective charge corresponding to the difference between its specific charge and the charge of the matrix. The resulting electrophoretic mobility will be modified according to that effective charge and result in the redistribution of the charge bands as compared to the pattern in a non-charged matrix. Proper choice of the constant charge allows the improvement of the spatial resolution of specific charge bands. Such a charged matrix can be used, for example, as a resolving gel when improved separation of closely spaced bands is required.

As used herein, “batch” refers to a quantity of anything produced at one operation, e.g., a quantity of a compound produced all at one operation.

In one aspect, the invention is a method of analyzing a LMWH preparation or mixture, including detecting the presence of a number of components, e.g., IIGHNAc, 6SICGHNS, 3S, 6S, I/GHNS, 6SGHNS, 3S, 6S, I/GHNAc,6SGHNS,3S, I/GHNS,6SI/GHNS,3S, I/GHNS,6SI/GHNS,3S,6S, I/GHNAc,6SGHNS,3S,I/GHNS, 6SI/GHNs, 3s or combinations thereof, as well as non-natural, that is, modified, sugars.

As used herein, “non-natural sugars” refers to sugars having a structure that does not normally exist in heparin in nature. As used herein, “modified sugars” refers to sugars derived from natural sugars, which have a structure that does not normally exist in a polysaccharide in nature, which can occur in a LMWH as a result of the methods used to make the LMWH, such as the purification procedure.

A further embodiment of the invention relates to the use of a method described herein for analyzing a sample, e.g., a composition including a mixed population of polysaccharides, such as glycosaminoglycans (GAGs), HLGAGs, UFH, FH, or LMWHs.

In some embodiments, the method further includes detecting one or more biological activities of the sample, such as an effect on cellular activities such as undesired cell growth or proliferation; cellular migration, adhesion, or activation; neovascularization; angiogenesis; coagulation; HIT propensity; and inflammatory processes. In some embodiments, the biological activity is anti-Xa activity; anti-IIa activity; the ratio between the anti-Xa activity and the anti-IIa activity; FGF binding; protamine neutralization; and/or PF4 binding.

Heparin (un fractionated heparin, UFH) and Low Molecular Weight Heparin (LMWH) elicit their anti thrombotic activity by two major mechanism, both involve binding of Antithrombin III (AT-III). In the first mechanism, the binding of Heparin to AT-III induce conformational change in AT-III that mediates inhibition of factor Xa. In the second, thrombin (factor IIa) binds to Heparin-ATIII complex to result in inactivation of thrombin. Standard Heparin test (such as, for example, activated partial thromboplastin time, aPTT, activated clotting time, ACT, and the like) mostly relay on the Anti factor IIa activity for their readout. Because the anti IIa activity of LMWH is lower than Heparin, these tests are less useful in measuring the biological activity of LMWH. Therefore, in order to test the biological activity of LMWH and LMWH fractions it is preferable to use the Anti-Xa as primary test and the specific Anti IIa as secondary test. Important LMWH features can thus be measured by the Anti-Xa/IIa activity ratio.

In some embodiments, the method may also include correlating one or more biological activities to the polysaccharide content of the sample. In some embodiments, the method may also include creating a reference standard having information correlating the biological activity to the specific identified polysaccharide. This reference standard can be used, e.g., to predict the level of activity of a sample, e.g., a LMWH preparation. Thus, in another aspect, the invention provides a method for predicting the level of activity of a LMWH preparation by analyzing the LMWH preparation and comparing the result to the reference standard described herein. The activity can be an effect on cellular activities such as cell growth or proliferation; cellular migration, adhesion, or activation; neovascularization; angiogenesis; coagulation; and inflammatory processes. In some embodiments, the activity is anti-Xa activity, anti-IIa activity, ratio between the anti-Xa activity and the anti-IIa activity; FGF binding, protamine neutralization, and/or PF4 binding.

In another aspect, the invention also provides a method of analyzing a sample of a heparin having a selected biological activity by determining if a component known to be correlated with the selected activity is present in the sample. The method can further include determining the level of the component. The activity can be an effect on cellular activities such as cell growth or proliferation; cellular migration, adhesion, or activation; neovascularization; angiogenesis; coagulation; and inflammatory processes, anti-Xa activity, anti-IIa activity, ratio between the anti-Xa activity and the anti-IIa activity; FGF binding, protamine neutralization, and/or PF4 binding.

In some embodiments, the biological activity-analysis information can be used to design a heparin, synthetic heparin, or LMWH preparation for a specific indication, e.g., renal impairment, autoimmunity, disease associated with coagulation, such as thrombosis, cardiovascular disease, vascular conditions or atrial fibrillation; migraine, atherosclerosis; an inflammatory disorder, such as autoimmune disease or atopic disorders; an allergy; a respiratory disorder, such as asthma, emphysema, adult respiratory distress syndrome (ARDS), cystic fibrosis, or lung reperfusion injury; a cancer or metastatic disorder; an angiogenic disorder, such as neovascular disorders of the eye, osteoporosis, psoriasis, and arthritis, Alzheimer's, or is undergoing or having undergone surgical procedure, organ transplant, orthopedic surgery, treatment for a fracture such as a hip fracture, hip replacement, knee replacement, percutaneous coronary intervention (PCI), stent placement, angioplasty, coronary artery bypass graft surgery (CABG). The specific indication can include cellular activities such as cell growth or proliferation; neovascularization; angiogenesis; cellular migration, adhesion, or activation; and inflammatory processes.

In another aspect, the invention relates to a method of making one or more specific batches of a polysaccharide preparation, wherein one or more of the polysaccharides of the batches varies less than a preselected preparation. In some embodiments, the method includes analyzing the polysaccharides of one or more batches of a product, according to the method of the present invention, and selecting a batch as a result of the determination.

Thus in another aspect the invention provides a method of analyzing a sample or a subject, e.g., a sample from a subject, for a heparin having anti-Xa activity, anti-IIa activity, the ratio between the anti-Xa activity and the anti-IIa activity, and the like, or any combination thereof. In some embodiments, the sample comprises a bodily fluid, e.g., blood or a blood-derived fluid, or urine. In some embodiments, the heparin comprises UFH or a LMWH, e.g., a LMWH having anti-Xa activity, anti-IIa activity, M118, M115, M411, M108, M405, M312, enoxaparin; dalteparin; certoparin; ardeparin; nadroparin; pamaparin; reviparin; tinzaparin, or fondaparinux. The method can include some or all of the following: providing a sample, e.g., from a subject, e.g., a human or veterinary subject or an experimental animal; determining if one or more components chosen from the group consisting of AUHNAc, 6sGHNs, 3s, 6s; AUHNs, 6sGHNs, 3s, 6s; AUHNAc, 6sGHNs, 3s; AUHNs,6sGHNs,3s or a fragment or fragments thereof is present in the sample; and optionally, measuring the level of the component or components. In some embodiments, the steps are repeated, e.g., at pre-selected intervals of time, e.g., every two to twenty-four hours, every four to twelve hours, every six to ten hours, continuous monitoring. In some embodiments, the method can also include establishing a baseline, e.g., a baseline for the component or components prior to the subject receiving the heparin.

In some embodiments, the human or veterinary subject is a patient with abnormal renal function as measured by RFI, urea, creatinine, phosphorus, GFR or BUN levels in blood or GFR or urine. In some embodiments, the human or veterinary subject has or is at risk for having complications associated with receiving heparin or LMWH, e.g., HIT. In some embodiments, the human or veterinary subject may be suffering from an immune deficiency, e.g., HIV/AIDS. In some embodiments, the human or veterinary subject is a pediatric patient. In some embodiments, the human or veterinary subject is pregnant. In some embodiments, the human or veterinary subject is a patient having a spinal or epidural hematoma. In some embodiments, the human or veterinary subject is a patient with a prosthetic heart valve. In some embodiments, the human or veterinary subject has an AT-III deficiency or abnormality. In some embodiments, the human or veterinary subject has a factor Xa deficiency or abnormality.

In another aspect, the invention relates to selecting a safer, less variable LMWH to use for treating a patient, by determining the polysaccharide content of a first batch of drug having a relatively high level of undesirable patient reactions, using the method of the present invention, determining the polysaccharide content of a second batch of drug having a relatively low level of undesirable patient reactions, and selecting a primary or secondary output correlated with the high or the low level of patient reactions. As used herein, “desirable patient reaction” refers to, inter alia, a preselected positive patient reaction as defined above. As used herein, “undesirable patient reaction” refers to an unwanted patient reaction, such as a negative patient reaction as defined above. As used herein, the term “treating” means remedial treatment, and encompasses the terms “reducing”, “suppressing”, “ameliorating” and “inhibiting”, which have their commonly understood meaning of lessening or decreasing.

In another aspect, the invention relates to a method of treating patients that have been excluded from LMWH treatment such as obese patients, pediatric patients, patients with abnormal renal function as measured by RFI, urea, creatinine, phosphorus, GFR or BUN in blood and urine and the interventional cardiology patient population by monitoring a subject receiving a polysaccharide, comprising monitoring the level of one or more of the components of the polysaccharide being administered.

In another aspect, the invention relates to a method of treating patients with complications of LMWH by monitoring a subject receiving a polysaccharide, comprising monitoring the level of one or more of the components of the polysaccharide being administered. In another aspect, the invention relates to the selection of a LMWH for treatment of a patient previously excluded from LMWH treatment because of an elevated risk of a negative patient reaction, by selecting a LMWH that has a low level or none of a primary or secondary output associated with a negative patient reaction.

According to some embodiments, the invention also relates to a method of determining the safety of compositions including a mixed population of polysaccharides, such as glycosaminoglycans (GAGs), Heparin like glycosaminoglycans (HLGAGs), UFH, FH, or LMWHs including but not limited to enoxaparin (Lovenox™); dalteparin (Fragmin™); certoparin (Sandobarin™); ardeparin (Normiflo™); nadroparin (Fraxiparin™); parnaparin (Fluxumm); reviparin (Clivarin™); tinzaparin (Innohep™ or Logiparinm), or Fondaparinux (Arixtra) in the treatment of subtypes of renal disease.

According to some embodiments, the invention also relates to a method for further understanding the mechanism of action of a specific heparin, LMWH or synthetic heparin and differentiating it from other heparins, LMWHs or synthetic heparins by analyzing and defining one or more of the heparins, LMWHs or synthetic heparins in a heterogeneous population of sulfated polysaccharides.

According to some embodiments, the invention further relates to a method for specifically identifying components of heparins, LMWHs or synthetic heparins which bind to proteins or other molecules which are associated with disease states or negative patient reactions, using, inter alia, chip-based specific affinity assays such as those disclosed for example in Keiser, et. al., Nat Med 7, 123-8 (2001). This chip-based approach to assess the binding of heparin fragments to various proteins may be readily used to assay an array of plasma and other proteins and assess binding properties.

According to some embodiments, the invention also relates to a method for broadening the therapeutic utility of heparins, LMWHs or synthetic heparins for use in areas other than as modulators of hemostasis, by understanding the mechanism of action of specific, individual components of specific heparins, LMWHs or synthetic heparins by analyzing, purifying and defining the specific components and the effect those components can have in the treatment of a specific disease.

According to some embodiments, the invention also relates to a method for broadening the therapeutic utility of heparins, LMWHs or synthetic heparins for use in areas other than as modulators of hemostasis, by designing compositions with enhanced activities for these diseases by analyzing, purifying and defining the activity of specific components and the effect those components can have in the treatment of a specific disease.

According to some embodiments, the invention also relates to broadening the therapeutic utility of heparins, LMWHs or synthetic heparins for treating clot bound thrombin by designing novel LMWHs of smaller sizes, and/or of increased anti-IIa activity that are active and can reach and treat the thrombus.

According to some embodiments, the invention also relates to a method for designing heparins, LMWHs or synthetic heparins with ideal product profiles including, but not limited to such features as high activity, having both anti-Xa and anti-IIa activity and the ratio thereof, titratable, well characterized, neutralizable, lower side effects including reduced HIT, attractive pharmacokinetics, and/or reduced PF4 binding that allow for optional monitoring and can be practically manufactured by analyzing, separating and defining the activity of specific components of a composition that includes a mixed population of polysaccharides. As used herein, “desired activities” refers to those activities that are beneficial for a given indication, e.g., a positive patient reaction as defined herein, inter alia. An “undesirable activity” may include those activities that are not beneficial for a given indication, e.g., a negative patient reaction, as defined herein, inter alia. A given activity may be a desired activity for one indication, and an undesired activity for another, such as anti-IIa activity, which while undesirable for certain indications, is desirable in others, notably acute or trauma situations, as discussed above.

According to some embodiments, the invention also relates to novel heparins made by the methods of the invention, e.g., novel heparins, LMWHs or synthetic heparins with desired product profiles including, but not limited to such features as high activity, both anti-Xa and anti-IIa activity and the ratio thereof, titratability, well characterized, neutralizable (e.g. by protamine), reduced side effects including reduced HIT, and/or attractive pharmacokinetics, that allow for optional monitoring, and novel heparins, LMWHs or synthetic heparins with different or enhanced anti-IIa activities. Thus in one aspect, the invention includes a LMWH preparation having an increased or decreased ratio of anti-IIa activity and anti-Xa activity, e.g., a LMWH preparation made by the methods described herein. In another aspect, the invention includes a panel of two or more LMWH preparations having different ratios of anti-IIa activity and anti-Xa activity, e.g., LMWH preparations made by the methods described herein.

According to some embodiments, the compositions of the invention may be derived from a natural source or may be synthetic. In some embodiments, the natural source is porcine intestinal mucosa.

According to some embodiments, the compositions may be formulated for in vivo delivery. For instance, the preparation may be formulated for inhalation, oral, subcutaneous, intravenous, intraperitoneal, transdermal, buccal, sublingual, parenteral, intramuscular, intranasal, intratracheal, ocular, vaginal, rectal, transdermal, and/or sublingual delivery.

Optionally, the compositions may also include one or more additives. Additives include, but are not limited to, dermatan sulfate, heparan sulfate or chondroitin sulfate.

In some embodiments of the invention, the preparation includes a specific amount of heparin. For instance the preparation may include 80-100 mole % heparin, 60-80 mole % heparin, 40-60 mole % heparin, or 20-40 mole % heparin. The heparin may, for example, be LMWH, native heparin, heparin sulfate, biotechnology-derived heparin, chemically modified heparin, synthetic heparin or heparin analogues.

In other aspects, the invention relates to a method for treating or preventing disease using different and specific novel LMWHs with specific product profiles at different phases in the course of treatment of a patient by dosing the patient with a LMWH having an enhanced activity for a specific disease state, e.g., a high level of anti-Xa and/or anti-IIa activity and than dosing with another LMWH composition having an enhanced activity for the changed disease state, e.g., having decreased PF4 binding.

In some aspects, the invention provides a method of treating a subject, e.g. a human or veterinary subject. The method includes some or all of the following: providing a panel of two or more LMWH preparations having different ratios of anti-IIa activity and anti-Xa activity; selecting a LMWH preparation having a desired ratio; and administering one or more doses of a therapeutically effective amount of the LMWH preparation to the subject.

It has also been discovered that polysaccharides having a low anti-Xa activity are particularly useful for treating atherosclerosis, respiratory disorder, a cancer or metastasis, inflammatory disorder, allergy, angiogenic disorder, and/or lung, kidney, heart, gut, brain, or skeletal muscle ischemial-reperfusion injuries. Respiratory disorders include but are not limited to asthma, emphysema, and adult respiratory distress syndrome (ARDS). Angiogenic disorders include but are not limited to neovascular disorders of the eye, osteoporosis, psoriasis, and arthritis. Thus, it is possible to tailor a compound which would be particularly useful for treating a subject that is preparing to undergo, is undergoing or is recovering from a surgical procedure or is undergoing a tissue or organ transplant. Surgical procedures include but are not limited to cardiac-pulmonary by-pass surgery, coronary revascularization surgery, orthopedic surgery, prosthesis replacement surgery, treatment of fractures including hip fractures, PCI, hip replacement, knee replacement, and stent placement or angioplasty.

It has also been discovered that a polysaccharide having a high anti-IIa activity has beneficial therapeutic properties; for instance, when delivered via a pulmonary delivery system, the rapid onset of action of polysaccharides having high anti-IIa activity is useful in treating acute conditions. Thus the instant invention relates to compositions with high anti-IIa activity for use in treatment of acute cardiac syndrome and myocardial infarction.

It was previously believed in the prior art that a high anti-IIa activity was not desirable for therapeutic purposes. As a result, polysaccharide preparations may have been selected based on a low anti-IIa activity. The compositions of the invention include polysaccharide compositions designed to have either a high or low anti-IIa activity. The compositions of the invention include polysaccharide compositions designed to have a high anti-IIa activity and sequence specific low anti-IIa activity and methods of using these compositions.

It had been found that some polysaccharides have therapeutic activity. In particular, heparin is a widely used clinical anticoagulant. Heparin primarily elicits its effect through two mechanisms, both of which involve binding of antithrombin III(AT-III) to a specific pentasaccharide sequence, HNAc/S, 6SGHNS, 3S, 6SI2SHNS, 6S contained within the polymer. First, AT-III binding to the pentasaccharide induces a conformational change in the protein that mediates its inhibition of factor Xa.

Second, thrombin (factor IIa) also binds to heparin at a site proximate to the pentasaccharide AT-III binding site. Formation of a ternary complex between AT-III, thrombin and heparin results in inactivation of thrombin. Unlike its anti-Xa activity that requires only the AT-111 pentasaccharide-binding site, heparin's anti-IIa activity is size-dependant, requiring at least 18 saccharide units for the efficient formation of an AT-III, thrombin, and heparin ternary complex. Additionally, heparin also controls the release of TFPI through binding of heparin to the endothelium lining the circulation system. Favorable release of TFPI, a modulator of the extrinsic pathway of the coagulation cascade, also results in further anticoagulation. In addition to heparin's anticoagulant properties, its complexity and wide distribution in mammals have lead to the suggestion that it may also be involved in a wide range of additional biological activities.

As detailed above, although heparin is highly efficacious in a variety of clinical situations and has the potential to be used in many others, the side effects associated with heparin therapy are many and varied. Side effects such as heparin-induced thrombocytopenia (HIT) are primarily associated with the long chain of unfractionated heparin (UFH), which provides binding domains for various proteins. This has led to the generation and utilization of low molecular weight heparin (LMWH) as an efficacious alternative to UFH. As a result, numerous strategies have been designed to create novel LMWHs with reduced chain lengths and fewer side effects. Of particular interest is the design of LMWHs that constitute the most active biological fragments of heparin. Examples of biologically active portions of a polysaccharide include but are not limited to a tetrasaccharide of the AT-III biding domain of heparin, a tetrasaccharide of the FGF biding domain of heparin,I/GHNAc, 6sGHNs, 3S, 6s, I/GUHs, 6sGHNs, 3s, 6s, I/GUHNAC, 6SGHNS,3S, I/GUHNS, 6SGHNS, 3s, or any combination thereof.

Sulfated polysaccharide preparations having structural and functional properties similar to LMWHs have been constructed and have been found to possess anti-Xa and anti-IIa activity as well as to promote the release of TFPI. Because of these attributes, the structure of these novel sulfated polysaccharide preparations could be assessed in conjunction with the beneficial activity.

In some embodiments, the method also includes monitoring the levels of LMWH in the subject, e.g., repeatedly monitoring the levels of LMWH in the subject over time. In some embodiments, the method includes adjusting the doses of the LMWH preparation. In some embodiments, the method includes monitoring the status of the subject in response to the administration of the LMWH preparation. In some embodiments, the method monitoring the status of the subject over a period of time. In some embodiments, the method also includes administering a different LMWH preparation based on changes in the status of the subject over time. In another aspect, the invention features a method of inhibiting coagulation in a patient by administering one or more doses of a therapeutic amount of a LMWH preparation described herein having high anti-Xa and anti-IIa activity, monitoring the status of the subject, then administering one or more doses of a therapeutic amount of a LMWH preparation as described herein having high anti-Xa activity alone.

In another aspect, the invention provides a method of treating a subject who has previously been diagnosed with HIT, comprising administering to the subject a therapeutically effective dose of a composition described herein having decreased PF4 binding activity.

### Inhibition of Tumor Necrosis Factor Activity

Tumor necrosis factor α (TNFα) is recognized as being involved in the pathology of many infectious and auto-immune diseases. Furthermore, it has been shown that TNF is the prime mediator of the inflammatory response seen in sepsis and septic shock, as well as in other conditions such as adult respiratory distress syndrome and graft-versus-host disease. TNF is also a key mediator in a number of autoimmune and inflammatory diseases such as rheumatoid arthritis, cerebral malaria and multiple sclerosis. Introduction of a humanized anti TNFα antibody (Infliximab) has been found to provide considerable relief to Inflammatory bowel disease (IBD) patients from disease symptoms, however serious toxicities related to the therapies have emerged and its safety profile is in doubt. TNFα level is upregulated and contributes to the pathogenesis of neurodegenerative diseases, such as Alzheimer's disease, multiple sclerosis, Parkinson's disease and the degeneration of the optic nerve in glaucoma. TNF-α is activating the glial cells which in turn secrete cytotoxic cytokines which lead to neuron and oligodendrocyte death.

Compositions according to the embodiments of the present invention may be used as effective anti-inflammatory agents useful to prevent or minimize a TNFα mediated condition.

As used herein “TNFα mediated condition” is intended to include a medical condition, such as a chronic or acute disease or pathology, or other undesirable physical state, in which a signaling cascade including TNFα plays a role, whether, for example, in development, progression or maintenance of the condition. Examples of TNFα mediated conditions include, but are not limited to: (A) acute and chronic immune, such as scleroderma, and the like; (B) infections, including sepsis syndrome, circulatory collapse and shock resulting from acute or chronic bacterial infection, acute and chronic parasitic infection, and/or infectious diseases, whether bacterial, viral or fungal in origin, such as a HIV or AIDS, and including symptoms of cachexia, autoimmune disorders, Acquired Immune Deficiency Syndrome, dementia complex and infections; (C) inflammatory diseases, such as chronic inflammatory pathologies, including sarcoidosis, chronic inflammatory bowel disease, ulcerative colitis and Crohn's pathology, and vascular inflammatory pathologies, such as, disseminated intravascular coagulation, and Kawasaki's pathology; (D) neurodegenerative diseases, including, demyelinating diseases, such as acute transverse myelitis; and lesions of the corticospinal system; and mitochondrial multisystem disorder; demyelinating core disorders, such as acute transverse myelitis; and Alzheimer's disease; (E) malignant pathologies involving TNF-.alpha. secreting tumors or other malignancies involving TNF, such as leukemias including acute, chronic myelocytic, chronic lymphocytic and/or myelodyspastic syndrome; lymphomas including Hodgkin's and non-Hodgkin's lymphomas; and malignant lymphomas, such as Burkitt's lymphoma or Mycosis fungoides; and (F) alcohol-induced hepatitis See, e.g., Berkow, et al., eds., The Merck Manual, 16.sup.th edition, chapter 11, pp 1380-1529, Merck and Co., Rahway, N.J., (1992).

### Matrix and Charged Separation Agents

A large number of methods and materials exist which enable the design of charged separation media with spatially distributed charge. Generally, all those methods are based on incorporation of anionic or cationic ion exchange resins at varying concentrations and compositions. The applicants of the present invention have discovered that these resins, when incorporated in and/or immobilized on matrices such as gels, porous glass, beads or viscous liquid compartments, create charged local environments like in ion exchange media. Designing the local concentration of the active ion exchange species according to the expected charge distribution of the analytes will provide the medium to separate and segregate the analytes according to their charge.

The matrix used in the present invention is preferably a low friction matrix. The mobility of analytes when driven by an electric field in a medium depends on the charge of the biomolecule and on the friction in the separation medium. Therefore, it is advantageous to minimize the friction component to reach the focusing (charge neutralization) position in a reason time. A “low friction matrix” as used herein is defined as a matrix in which the friction coefficient is comparable to the friction coefficient in a 4% polyacrylamide or lower. Friction coefficients of polyacrylamide gels are routinely know to a person of skill in the art. Ranges of translational friction coefficient can be derived from published art on mobilities and viscosities of various concentration gels, as is known to a person of skill in the art.

The matrix can comprise low density solid gels like polyacrylamide or agarose which can incorporate the charged separation agents. Alternatively, liquid matrices may be used which are capable of incorporating the charged molecules. Such liquids can be for example very low concentration (e.g. 1%) polyacrylamide which can copolymerize with immobilines. Another possibility is linear polymers, which due to the lack of cross linking behave like a viscous liquid. Another embodiment can be mixtures of non charged liquids (water) and polymer beads incorporating the charged separation agent (e.g., custom prepared ion exchange beads, polyacrylamide beads with immobilines, etc.). Since the charge neutralization occurs in the beads only their density should be high enough to stop all the biomolecules drifting through the medium.

When the charged matrix is either a highly viscous liquid or a solid liquid mixture (beads) there is a need to contain the medium representing a specific charge density in a compartment isolated from its neighbor compartments to prevent mixing. This is achieved by placing the charged medium between separators comprising uncharged membrane. Such a membrane should allow the transport of the charged biomolecules but prevent intermixing of the content of each compartment. The material of the uncharged membrane can be a polymeric membrane like agarose, polyacrylamide, cellulose etc. It should be as thin as possible to minimize the drift time and still support the content of the compartment. The separation medium (liquid or liquid-bead mixture) is preferably not immobilized on the membrane. The membrane serves only as a physical separator.

Examples of such ion exchange materials suitable as the charge separation agents include various organic ion exchange resins composed high molecular weight polyelectrolytes. Non-limiting examples of suitable ion exchange resins are:

1) Dowex 66 Anion-Exchange Resin

2) Dowex1-X2 (AG 1-X2) Anion-Exchange resin

3) Dowex 1-X4 (AG 1-4X) Anion-Exchange resin

4) Dowex 1-X8 (AG 1-4X) Anion exchange resin

5) AG MP-1

6) Amberlite™ IRA-401

7) Amberlite™ IRA-402

8) Amberlite™ IRA-400

9) Amberlite™ CG-400

10) Amberlite™ IRA-904

11) DUOLITE®¹¹³

12) DUOLITE®^{A161}

13) Dowex Ion Exchange Resin^{2-X8 (AG2-x8)}

14) Dowex Ion Exchange Resin^{2-X10 (AG 2X10)}

15) Amberlite™ IRA-410

16) DUOLITE^{A 116}

17) DUOLITE®^{A162}

18) Biorex 9

19) Biorex 5

20) DUOLITE^{A 303}

21) DUOLITE^{A378}

22) Dowex Ion Exchange Resin^{3-X4A (AG 3-X4A)}

23) Amberlite™ IR-45

24) amberlit IRA-67

25) Amberlite™ IRA-93

Another example of suitable separation agents are acrylamido buffers used for preparation of Isoelectric Focusing Strips (immobilines). Immobilines are acrylamide derivatives that are weak acids or weak bases, and have the general structure CH₂═CH—CO—NH—R, where R contains either a carboxyl or an amino group.

Another way of preparing stable charge density gradients in gel matrices is by incorporating (polymerizing, immobilizing) polypeptide sequences in the gel by methods known from affinity gel electrophoresis (using, for example, hemoglobin, lectin etc.).

For negatively charged gradients, it is also possible to utilize immobilized DNA fragments.

Desired charge densities can be obtained by using any of these reagents, alone or in any combination.

It should be apparent to a person of skill in the art that the present invention is not limited to the use of the above described reagents, and that any other reagent capable of creating a stable charge gradient across a polymeric gel or other porous matrix can be used in the methods and systems of the present invention.

The principles of embodiments of the present invention differ significantly from the principle of ion exchange chromatography. Ion exchange chromatography is based on the amphoteric property of proteins and the specific protein charge is determined by the pH of the buffer solution which contains the protein mixture. The ion exchange column is designed to trap by charge neutralization that specific charged protein while all other proteins pass through the column. Therefore, according to the present invention, a charge gradient medium is used, and the analytes are neutralized by their charge independent of the buffers.

The amount of charged separation agent to be included in the matrix will vary depending on the type of analyte being separated. For example, if the analyte has a high molecular weight, a larger amount of separation agent will typically be used to achieve adequate separation. If the analyte has a low molecular weight, lower amounts of the separation agents will be used. Generally, in order to generate an immobilized charge density gradient in a separation medium, one has to calculate the required concentration of the charge generating species to be incorporated in the gel at each point of the gradient. The charge density is estimated from the concentration and the dissociation constant of the charging compound. Table 1 below presents some examples of a design of a concentration gradient in a polyacrylamide gel by utilizing an immobiline charged separation agent with pH=9.3. The known dissociation constant for this particular immobiline is ˜0.1 at room temperature.

### Matrix Systems

In one embodiment, the present invention provides systems that can be used to separate analytes based on their charge. In one currently preferred embodiment, the matrix is a polymeric gel. In accordance with this preferred embodiment, the gels of the present invention are polymeric gels which have been modified to include a charged separation agent. The gels contain charged regions that result in a charged density gradient, which can be continuous or discrete, distributed across the gel. Preferably the charge gradient is created from a low charge to a high charge.

Any type of polymeric gel known in the art can be used in the methods and systems of the present invention. In accordance with a preferred embodiment, the polymeric gel is a polyacrylamide gel. However, other gels can also be used, for example agarose gels, composite polyacrylamide-agarose gels, gelatin, and the like.

Another matrix suitable for this invention are viscous liquids like for example very low density polyacrylamide or other matrices in which charged separation agents can be incorporated.

Suitable gels for this type of separation include but are not limited to low percentage polyacrylamide (e.g., about 5% or less) or composite acrylamide agarose gels (about 2%-5% acrylamide and about 0.5%-1% agarose). The latter gel system permits the use of very low percentage polyacrylamide as the sieving matrix and substrate for covalently bonded charged species while the agarose provides mechanical support.

Suitable gels for use in the methods of the present invention include, but are not limited to, slab gels, planar gels, capillary gels, in-tube gels, discrete gel lanes in channels, separation columns or any other geometry which preserves the charge distribution. This will enable a design where the linear charge resolution can be optimized for different charge regions.

For preparing the modified gels of the present invention, the charged separation agent is typically mixed with the rest of the constituents of the gel, and polymerization and casting of the gel is carried out as known to a person of skill in the art for each gel system.

In the case of a polyacrylamide gel, the method of preparation of a slab gel with a built-in charge gradient can be prepared by a standard method of casting of gels analogous to the preparation of Immobilized pH Gradient (IPG) strips with the appropriately designed quantities of ion exchange resin or immobiline. The preparation of IPG strips has been described in, for example in: ELECTROPHORESIS IN PRACTICE by Reiner Westermeier, Second Edition, VCH, 1997, the contents of which are incorporated by reference herein.

The matrices of the present invention can be in the form of a thin or thick planar film gel, typically having a thickness ranging from 0.5 mm to 3 mm, and dimensions of typically from 2 cm×3 cm up to 18 cm×20 cm, they can be filled in a capillary or tubes typically having a thickness of about 50-500 μm, for example 100 μm, 75 μm and 50 μm or they can be in the form of a single or multiple channels with cross section of 100 microns×100 microns or 1 mm×1 mm and length of 1 cm up to 20 cm.

Advantageously, the matrices according to embodiments of the present invention can be applied and extended to multi array systems such as serial arrays of discrete compartments with charge density overlapping a specific charge range bridged by a low friction medium, arrays in a chip format, pre-designed charge focusing arrays for specific polysaccharide charge in application for diagnosis, multi compartment trapping devices for specific charge ranges suitable for fractionation of complex samples and amenable for scale up (purification) and other separation systems using other low friction media, under widely different conditions. For example, based on the selective trapping (focusing) capability of the charged gels, one can construct a multicompartment system which will fractionate a complex sample by trapping samples in specific compartments according to their charge.

The chip-like device, in one embodiment, comprises discrete channels of charged gels each pixel possessing a charge density for focusing of a specific charge. The discrete pixels can be serially interconnected with a low friction uncharged gel (for example agarose) bridge or with liquid interconnects. The chip device can be automated using automation techniques commonly known in the art.

Such an interconnected linear array will cover a specific charge range with the pre-determined charge resolution. Parallel positioned linear arrays, each corresponding to a different charge interval will result in a 2D array covering a desired charge range.

Using the same configuration it is possible to design a chip which will focus only predetermine polysaccharides for diagnostic purposes. If employed with fluorescent color markers or radiolabeled markers, such a chip will enable fast readout for detection of specific polysaccharide markers.

According to some embodiments, the invention can also be used to focus polysaccharide-antibody complexes in pre-designed compartments for diagnostic applications.

Aside from polymeric gels (e.g., polyacrylamide gels, agarose gels and composition polyacrylamide-agarose gels), other suitable media or substrates for use in the methods of the present invention are media on which charged anionic or cationic species can be immobilized, such as porous glass, high viscosity liquid polymers, polymeric beads etc.

The compound used according to the invention can be formulated by any required method to provide pharmaceutical compositions suitable for administration to a patient.

The novel compositions contain, in addition to the active ingredient, conventional pharmaceutically acceptable carriers, diluents and the like. Solid compositions for oral administration, such as tablets, pills, capsules or the like, may be prepared by mixing the active ingredient with conventional, pharmaceutically acceptable ingredients such as corn starch, lactose, sucrose, sorbitol, talc, stearic acid, magnesium stearate, dicalcium phosphate and gums, with pharmaceutically acceptable diluents. The tablets or pills can be coated or otherwise compounded with pharmaceutically acceptable materials known in the art to provide a dosage form affording prolonged action or sustained release. Other solid compositions can be prepared as microcapsules for parenteral administration. Liquid forms may be prepared for oral administration or for injection, the term including subcutaneous, intramuscular, intravenous, and other parenteral routes of administration. The liquid compositions include aqueous solutions, with or without organic cosolvents, aqueous or oil suspensions, emulsions with edible oils, as well as similar pharmaceutical vehicles. In addition, the compositions of the present invention may be formed as encapsulated pellets or other depots, for sustained delivery.

Each of the limitations of the invention can encompass various embodiments of the invention. It is, therefore, anticipated that each of the limitations of the invention involving any one element or combinations of elements can be included in each aspect of the invention.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Separation of LMWHs

LMWH fragments were separated using gradient charged electrophoresis resolving gel in the following manner:

Gradient charged resolving gel was prepared by mixing two solutions with different concentration of immobiline buffer (IMB) (table 4) using gel casting gradient mixer. The gel casting gradient mixer was loaded with the IMB solutions: L-IMB gel solution (2 ml) was added in to the reservoir chamber and D-IMB as a heavy solution (2 ml) was added in to the mixing chamber with magnetic stirrer stirring at a moderate speed. Ammonium persulphate (15 μl of 40%) was added into each chamber and the solutions were pumped into the gel caster using a gradient pump. The gel was left to polymerize (20 minute at RT and then 1 hour at 50° C.). Following polymerization the gel was cooled down (1 h RT and then 1 h 4° C.). A Mylar film [Gel Bond, 4.5% T polyacrylamide (3.3% cross-linker) in the presence of a gradient of positively charged Immobiline] was used as support for easy handling when opening the cassette.

TABLE 4

| D-IMMOBILINE BUFFER (IMB) | | L-IMMOBILINE BUFFER (IMB) | |
|---|---|---|---|
| 30% BIS- | 4.5% | 30% BIS- | 4.5% |
| ACRYLAMIDE | | ACRYLAMIDE | |
| GLYCEROL | 17.4% | GLYCEROL | 0% |
| DISTILLED | UP TO 10 ML | DISTILLED | UP TO 10 ML |
| WATER | | WATER | |
| GEL BUFFER* | 122 mM | GEL BUFFER | 122 mM |
| IMB | 10 mM | IMB | 0 mM |
| TEMED | 5 μl | TEMED | 5 μl |
| TOTAL | 10 ML | TOTAL VOLUME | 10 ML |
| VOLUME | | | |

*Gel buffer Solution - 0.48 M Tris/Acetate pH-6.4: TRIZMA (Tris[hydroxymethyl]aminomethane), Acetic acid, Water 18 megohm. Filtrate through 0.2 μm filter. Store in 4° C.

LMWH samples (Enoxiparin and Tinzaparin) were applied directly on the gel by using whatman paper (pieces of a size 3×3 mm) and placed on the gel surface a few mm from the cathodic buffer strip. The gels were run horizontally in a multiphor II Chamber (GE Healthcare, 10° C., 300V, 12 mA, 3W, −1.5 h) using cathode buffer (0.1M Tris, 0.1M Tricine, TIZMA (Tris[hydroxymethyl]aminomethane, TRICINE (N-[Tris(hydroxymethyl)methyl]glycine, Water 18 megohm, filtered through 0.2 μm filter, 4° C.), and anode buffer (0.1M Tris, 0.1M Acetate, TIZMA (Tris[hydroxymethyl]aminomethane, Acetic acid pH 6.4, Water 18 megohm, filtered through 0.2 μm filter, 4° C.). Following electrophoresis, the gels were stained for the detection of Heparin using “stains-all” staining and photographed.

Using the gradient charged electrophoresis resolving gel samples of LMWH (Enoxiparin and Tinzaparin) were separated. The LMWH samples (Enoxiparin (lanes 1-2 in FIG. 1) and Tinzaparin (lanes 3-4 in FIG. 1)), were separated into 12-14 distinct fractions, as shown in FIG. 1. The gradient charged electrophoresis has allowed achieving superb resolution of LMWH, due differences in charge distribution along different subpopulations of LMWH and some differences in their chemical composition. Comparable experiment done using capillary electrophoresis failed in separation of the fragments in Enoxiparin and Tinzaparin samples.

### Example 2

### Separation of LMWH Fractions Obtained from Size Exclusion Chromatography (SEC)

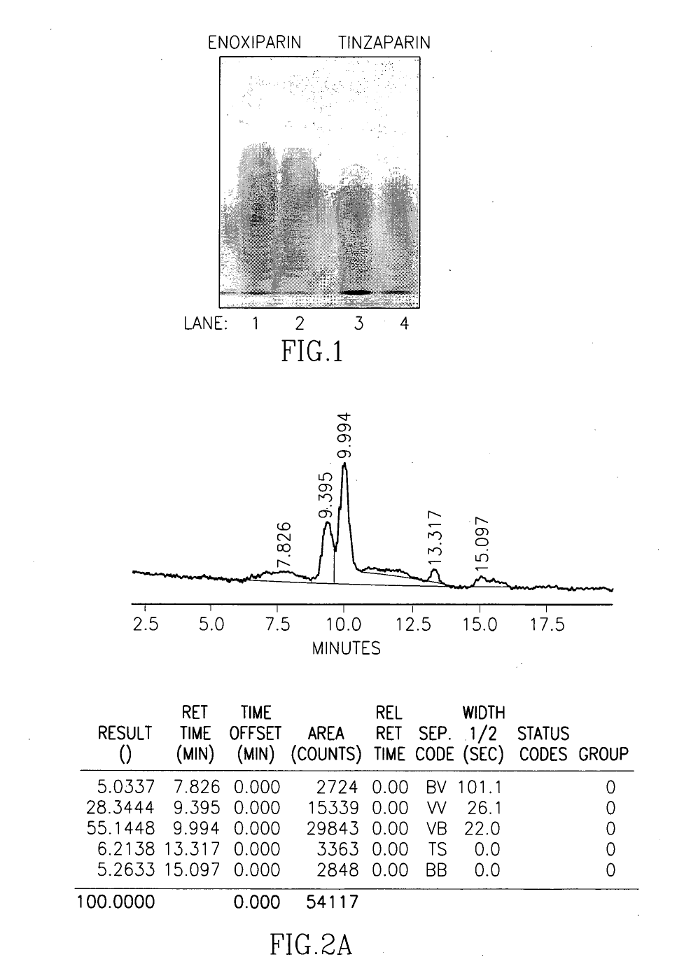

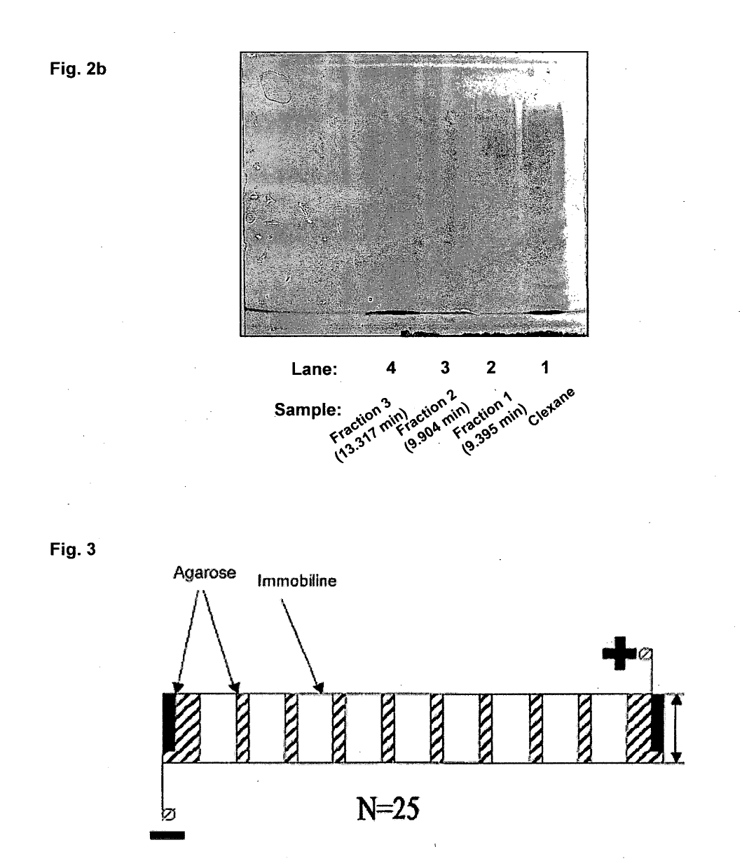

Enoxiparine (Clexane) was separated by size exclusion chromatography (SEC) using HPLC (Varian Pro Star) with UV detector tuned at 232 nm. A TSK G3000PW (Tosoh) column, at flow rate of 0.8 ml/min, in 75 mM ammonium bicarbonate was employed. The separation was monitored by UV absorbance at 232 nm. The results of this separation are presented in FIG. 2a, representing bands of different polysaccharide size. Six fractions were collected and concentrated by freeze-drying.

Three pronounced fractions (9.395, 9.994 and 13.317) were analyzed using polycationic gels prepared as described in Example 1 hereinabove (nonlinear gradient 0-10 mM). The resolving gel and lower running buffer used are 0.48 M Tris/Acetate pH −6.4. The upper running buffer is 0.1M Tris, 0.1M Tricine. Electrophoretic separation was performed in the gel at 300V during 4 h. After electrophoresis bands were visualized by staining with Stains-ALL stain.

The separation pattern of the three SEC fractions (9.395, 9.994 and 13.317) is presented in FIG. 2b (lanes 2, 3 and 4, respectively). As shown, the pattern of each fraction (lanes 2-4) consists of a large number of bands, each band representing a specific charge of a polysaccharide molecule. In lane no. 1, the separation pattern of Clexane is shown. Thus, the results show that by using a separation method according to embodiments of the present invention, each single SEC fraction (9.395, 9.994 and 13.317, lanes 2-4, respectively), in fact include several charged polysaccharide molecules. Hence, the separation method, according to embodiments of the invention provides an evidently more subtle and fine separation as compared to other separation methods.

### Example 3

### Biological Activity of Specific Charge Polysaccharide Fractions

The process of separation of specific charge polysaccharides using methods according to embodiments of the invention are scaled up to obtain quantities of fractions which are further tested for biological activity both as single fractions or combination of fractions to determine and to construct effective compositions. To this aim, various methods are employed. Heparin (un fractionated heparin, UFH) and Low Molecular Weight Heparin (LMWH) elicit their anti thrombotic activity by two major mechanism, both involve binding of Antithrombin III (AT-III). In the first mechanism, the binding of Heparin to AT-III induce conformational change in AT-III that mediates inhibition of factor Xa. In the second mechanism, thrombin (factor IIa) binding to Heparin-ATIII complex results in inactivation of thrombin. Standard Heparin tests (for example, activated partial thromboplastin time (aPTT), activated clotting time (ACT)) mostly relay on the Anti factor IIa activity for their readout. Because the anti IIa activity of LMWH is lower than Heparin, these tests are less useful in measuring the biological activity of LMWH. Therefore, in order to test the biological activity of LMWH and LMWH fractions it is preferred to use the Anti-Xa as primary test and the specific Anti IIa as secondary test.

The anti factor Xa activity of LMWH fractions is determined by testing the sample potentiating effect on antithrombin (ATIII) in the inhibition of factor Xa. Anti factor Xa activity is indirectly measured (for example, by using a Diagnostica Stago analyzer with a Stachrom® Heparin test kit; By using an ACL Futura™ Coagulation system with the Coatest® Heparin kit from Chromogenix; or any desirable equivalent system).

The anti factor IIa activity is determined by testing the sample potentiating effect on antithrombin (ATIII), in the inhibition of thrombin. The anti factor IIa is measured, (Diagnostica Stago analyzer on an ACL Futura™ Coagulation system with reagents from Chromogenix (S-2238 substrate, Thrombin and Antithrombin) or any equivalent system.

Both methods of activity analysis are calibrated using the NIBSC International Standard for Low Molecular Weight Heparin.

An important LMWH feature can thus be measured by the Anti-Xa/IIa activity ratio. The ratio of anti factor Xa to anti factor IIa activity is calculated by dividing the anti factor Xa activity by the anti factor IIa activity.

The level of LMWH anti Xa and/or anti IIa activity naturalization by protamine sulfate is also measured by administration of commercially available protamine sulfate followed by measuring LMWH activity.

### Example 4

### Gel Multicompartment Fractionation Device

The following example demonstrates an application of the methods according to some embodiments of the invention. Based on the selective trapping (focusing) capability of the charged gels, one can construct a multicompartment system which will fractionate a mixture of polysaccharides in specific compartments according to their charge.

The device is constructed as a serial system of immobiline gel membranes in increasing order of Immobiline concentration, each membrane separated from its neighbour by a low density agarose gel partition. For this example a device was prepared with 25, 1.5 mm thick 4% polyacrylamide immobiline compartments, arranged in a steplike gradient of immobiline concentration and interspaced with and 1% agarose membrane 0.2 mm thick. The steplike gel-immobiline gradient was prepared by pouring and polymerizing the PA gel solutions in fauns created by the agarose membranes. The compositions and polymerization procedures were like shown in the previous examples. A schematic illustration of the device is shown in FIG. 3

### Example 5

### Liquid Multicompartment Charge Fractionation Device

This example demonstrates the performance of a multicompartment fractionation device in which the charge neutralization medium consists of a viscous liquid in the form of a 1% Polyacrylamide with immobilines and is used for fractionation of a mixture comprising polysaccharides.

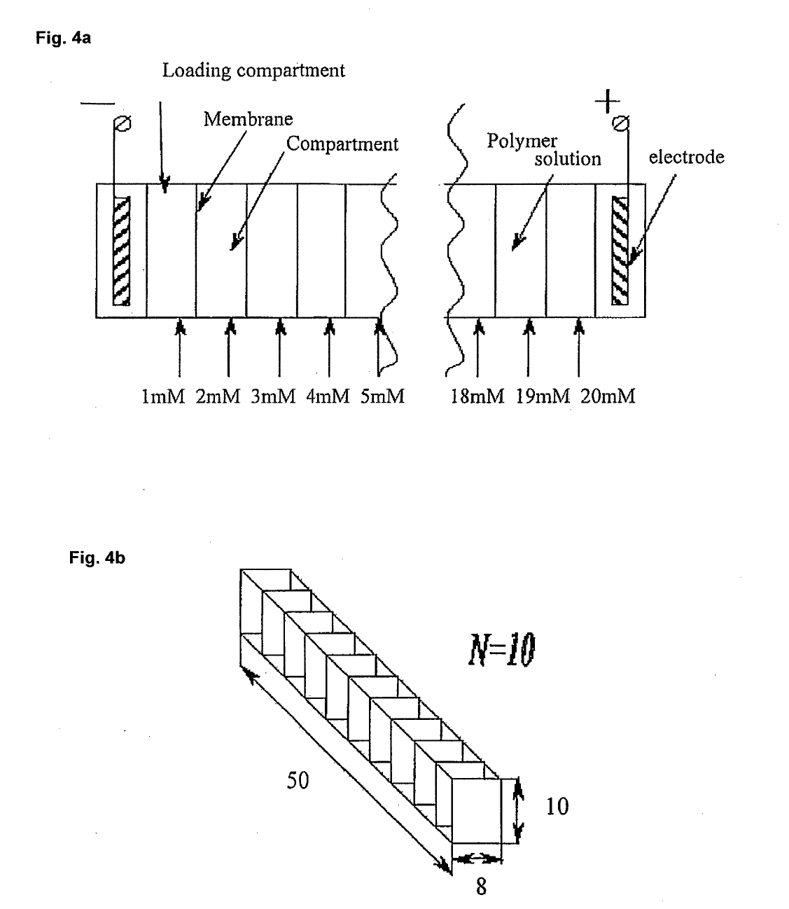

The device was constructed as presented in FIGS. 4a and 4b with the following materials:

Compartment wall—a 15% PAAG (0.75 mm-thickness);

Compartment material: 1% Polyacrylamide with immobilines

The starting materials were:

Acrylamide (Cat.N. 161-0108 BioRad); Immobiline (Immobiline buffer pKa 10.3 (Cat no 01741, Fluka)); TEMED (Cat N 161-0800, Bio-Rad);
Ammonium Persulfate (Cat N161-0501, Bio-Rad); sodium dodecyl sulfate (Cat. N L3771, Sigma).

### The Composition of the 1% Polyacrylamide was as Follows:

| | |
|---|---|
| Acrylamide | 1.0% |
| dist. Water | |
| Gel buffer | 122 mM |
| SDS | 0.10% |
| Immobilines 10.3 | 0-20.0 mM |
| Ammonium persulfate | |
| TEMED | |

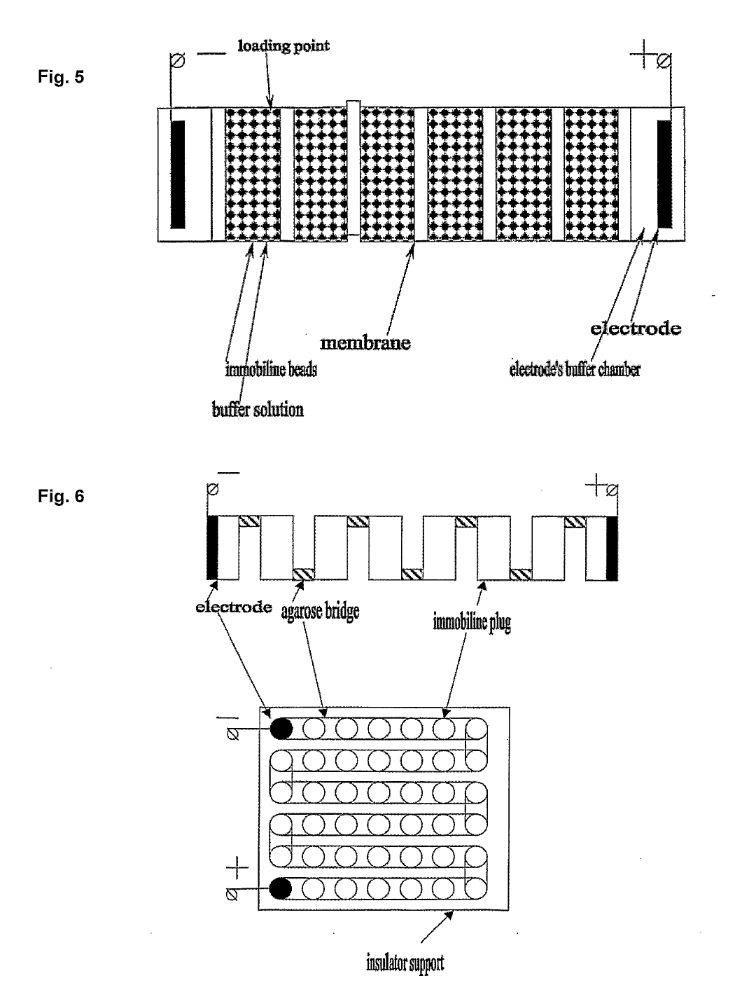

Another set-up showing an alternative embodiment of the multi compartment system is illustrated in FIG. 5.

### Example 6

### Multi-Compartment Charge Fractionation Chip

This example demonstrates the concept of the multi-compartment chip representing an important application of the methods of invention. A multi compartment chip was prepared according to the design as shown, for example, in FIG. 6. 70 holes of 1 mm-diameter and; 1 mm-length were machined in a PMMA slab. Each hole was filled with a 4% polyacrylamide immobiline solution to create a serial step like gradient of immobiline concentration (0-35 mM). The resulting PA immobiline plugs were interconnected by 1% agarose bridges.

Immobiline buffer pKa 10.3 (Cat no 01741, Fluka) was used for creation of the immobiline gradients. Immobiline gradient solutions were prepared as in previous examples.

### Example 7

### Effects of LMWHs on the Development of Inflammatory Bowel Disease In Vivo

The aim of the study is to evaluate the inhibitory effects of LMWH purified according to the present invention on the development of inflammatory bowel disease (IBD) in mice models.

Acute IBD is generated in BALB/C mice (6 mice per group) anesthetized with Ketamine & Xylazine, by DSS administered via the drinking water (3.5% w/v) for 7 days. LMWH preparations are administered to these animals intraperitoneally at doses of 25 and 75 μg/mouse beginning 48 hrs prior to initiation of DSS administration and at 48 hr intervals thereafter. After 16 days the mice were sacrificed with high dose of sodium pentobarbital, the gastro-intestinal tract removed, its overall length measured and evaluated compared to control untreated healthy mice.

### Example 8

### Preventing the Cell Death Induced by TNFα Using LMWHs

The aim of the study is to evaluate the ability of LMWHs purified according to the present invention to salvage non malignant cells from death induced by TNFα.

Mouse L cells (ATCC) are cultured in complete MEM medium in 37° C. incubator with 5% CO₂ and 95% humidity. The culture cells are divided to several groups for control, several types and concentrations of LMWHs with or without TNFα.

LMWH preparations or control samples are applied to the cells 48 hr prior to TNFα administration. The experiment was terminated 24 hrs after TNF administration and cell viability evaluated by MTT assay.

### Example 9

### Preventing the Cell Death Induced by TNFα Using Hypericin—Evaluation with the Hemacolor Assay

The aim of the study is to evaluate the ability of LMWH preparations prepared according to the method of the present invention to salvage non malignant cells from death induced by TNFα using an alternative method of quantification—Hemacolor assay.

Mouse L cells (ATCC) are cultured in complete MEM medium in 37° C. incubator with 5% CO₂ and 95% humidity. The culture cells are divided to several groups for control, several types and concentrations of LMWHs with or without TNFα. LMWH preparations are applied to the cells 48 hr prior to TNFα administration. The experiment was terminated 24 hrs after TNF administration and cell viability evaluated using the Hemacolor assay.

### Example 10

### Effects of LMWHs on the Development of Inflammatory Skin Reactions Induced by Herpes Simplex Type 1 Virus in Guinea Pig Dorsa

The aim of the study is to evaluate the inhibitory effects of LMWH preparations prepared according to the method of the present invention on the development of inflammatory erythema and edema following infection with herpes virus.

Male guinea pigs are anesthetized with Ketamine 100 mg/ml and Xylazine 20 mg/ml (7:3), total volume of 0.5 ml/kg. Six small 4 mm crossed incisions are made in the skin. Herpes simplex type 1 virus at a titer of 10⁶ TCID/ml (Tissue culture infective dose) is applied to 4 of the 6 incisions. The two others served as controls for incision-induced mechanical inflammation in the absence of virus (controls, not infected with a virus). Several preparations and concentrations of LMWH are applied topically 3× per day for 3 consecutive days and the animals evaluated for inflammation related symptoms after 96 hrs.

While the certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1. A method for the separation or analysis of polysaccharides according to their charge, comprising the steps of:
i. providing a preparation comprising at least one charged polysaccharide;
ii. a. subjecting the preparation to an electric field using a matrix comprising a charged separation agent, or
b. contacting said preparation with a matrix comprising a charged separation agent having an opposite charge to that of the at least one polysaccharide; wherein the charged separation agent is distributed throughout the matrix so as to create a charge density gradient, and applying an electric field across said matrix;
wherein the charged separation agent is distributed throughout the matrix so as to create a charge density gradient,
thereby separating said polysaccharides according to their charge.

2. The method according to claim 1, wherein the matrix is selected from the group consisting of a porous matrix, a polymeric gel, porous glass, high viscosity liquid and polymeric beads.

3. (canceled)

4. The method according to claim 2, wherein the high viscosity liquid and polymeric beads are separated in compartments by a porous membrane.

5. The method according to claim 2, wherein the polymeric gel is a polyacrylamide gel.

6. The method according to claim 1, wherein the charge gradient is from a low charge density to a high charge density.

7. The method according to claim 1, wherein the charge separation agent is evenly distributed throughout the matrix.

8.-10. (canceled)

11. The method according to claim 1, wherein said separation agent is an ion exchange resin or an immobiline.

12. (canceled)

13. The method according to claim 1, wherein said polysaccharides comprise at least one polysaccharide selected from the group consisting of heparin, heparin fragment, and low molecular weight heparin.

14. (canceled)

15. The method of claim 1, further comprising extracting of the polysaccharide from the matrix.

16. (canceled)

17. A polysaccharide separated according to the method of claim 1.

18. The polysaccharide according to claim 17 which is a low molecular weight heparin (LMWH).

19. A preparation comprising at least one LMWH separated or analyzed according to the method of claim 1.

20. A pharmaceutical composition comprising as an active ingredient at least one LMWH separated according to the method of claim 1.

21.-28. (canceled)

29. A method for prevention or inhibition of a TNFα-mediated disease or condition comprising administering to a patient in need thereof a therapeutically effective amount of a LMWH according to claim 18.

30. The method according to claim 29, wherein the TNFα-mediated disease or condition is selected from the group consisting of: inflammatory bowel disease, ulcerative, acute or ischemic colitis, Crohn's disease, cachexia (wasting syndrome), septic shock (sepsis, endotoxic shock), disseminated bacteremia and a neurodegenerative disorder.
